(19) United States
(12) Patent Application Publication
(10) Pub. No.: US 2020/0368062 A1
(43) Pub. Date: Nov. 26, 2020

(54) Selective brain cooling system

(71) Applicant: LETO SOLUTIONS, INC, San Antonio, TX (US)

(72) Inventors: Sean T. Baker, San Antonio, TX (US); Gary Walters, San Antonio, TX (US)

(21) Appl. No.: 15/999,334

(22) PCT Filed: Feb. 19, 2017
(86) PCT No.: PCT/US2017/018534

§ 371 Date: Nov. 26, 2019

Related U.S. Application Data
(60) Provisional application No. 62/297,784, filed on Feb. 19, 2016.

(51) Int. Cl. A61F 7/02; A61F 7/00

(52) U.S. Cl. CPC A61F 7/02; A61F 7/007; A61F 2007/0012; A61F 2007/0247; A61F 2007/0095; A61F 2007/0228; A61F 2007/0075

(57) ABSTRACT

A selective brain cooling system for controlling the temperature of the brain of a patient, the system comprising a headliner and/or neck brace, and one or more thermoelectric cooling elements.

### CROSS-REFERENCE TO RELATED APPLICATION

This application claims priority to U.S. Provisional Patent Application 62/297,784 entitled “Selective Brain Cooling System” filed Feb. 19, 2016, which is hereby entirely incorporated herein by reference.

### FIELD

The disclosed subject matter is directed to a selective brain cooling system that relates to cooling the head, neck, and/or brain of a patient. The disclosed subject matter relates to inducing cerebral hypothermia for treating traumatic brain injury, mild traumatic brain injury, stroke, heat stroke, aneurysms, insomnia, and other brain injuries or conditions.

### BACKGROUND

Therapeutic hypothermia refers to the deliberate reduction of body temperature to achieve a therapeutic goal. This can refer to “core cooling”, in which the goal is to reduce core body temperature in an attempt to reduce the body's demand for oxygen. It can also address Selective Brain Cooling (SBC), which is employed when the goal is to cool the brain, but maintain core body temperature at normal levels. In some cases, therapeutic hypothermia is used to slow down cellular metabolism, as is the case with traumatic brain injury; in other cases, such as after an hypoxic event, it is used to prevent or minimize cell death.

Cerebral therapeutic hypothermia or SBC is a method used in patients who have suffered a traumatic brain injury (TBI) to mitigate the inflammatory response, as well as to stabilize the blood brain barrier and reduce cerebral edema. This facilitates the reduction in cell death which helps to reduce neurological injury. SBC is also a therapy used in patients who have experienced an hypoxic event, such as birth asphyxia, stroke, or epilepsy. In these cases, SBC serves to slow down cellular metabolism, which reduces the brain's need for oxygen—also with the goal of reducing or limiting neurological injury.

Head trauma (mild, moderate, or severe), stroke, cardiac arrest, cerebral aneurysms, neonatal hypoxia, and epilepsy are among the conditions which can cause temporary or permanent, mild or severe, brain impairment. Prompt action after the injury has occurred plays a critical role in minimizing the impairment. Research indicates that cooling therapy should be ideally maintained for a period of at least 72 hours.

A cerebrovascular accident (CVA), or stroke, is the loss of brain function due to a disrupted supply of oxygen to the brain. Strokes are commonly caused by either a hemorrhage resulting from a blocked or ruptured blood vessel, or a blood clot. Another cause but less frequent is ischemia or a loss of oxygen to local areas of the brain. When the brain is deprived of oxygen the affected area will suffer neurological damage on a time dependent basis; the longer the cells are deprived of oxygen, the more severe and permanent the neurological damage. Cerebrovascular accidents are the number two cause of death worldwide and the leading cause of adult disability in the US.

If the patient receives prompt medical care, there are drugs that can be administered to mitigate the neurological effects of stroke. Current research suggests use of a brain cooling system to induce local hypothermia in the stroke affected area and deep brain. By inducing local hypothermia, the cellular metabolism is lowered and thus the oxygen demand is lowered. The brain's damage following a stroke is critically dependent upon the amount of time the brain tissue is oxygen deprived. For the brain cooling treatment to be effective, the brain should be cooled as soon as possible, optimally within three hours after the injury. After three hours the neurological damage will likely be irreversible.

Birth asphyxia occurs when an infant's brain and body is deprived of oxygen during the birthing process. If left untreated, asphyxia can result in serious and permanent brain damage, severe cerebral palsy, and even death. SBC has been researched as a treatment for birth asphyxia (also referred to as hypoxic-ischemic encephalopathy—HIE) for more than 20 years, and recent evidence suggests it is effective in reducing the incidence and severity of brain damage. HIE is a progressive injury that begins shortly after oxygen deprivation and may continue for 48 hours. Hearing loss, learning disability, motor dysfunction, cerebral palsy, and death can result from HIE. Clinical research from the UK reports that, in a study of infants who suffered birth asphyxia, the percentage of those who survived with no sign of brain damage increased to 44% when cooling was employed, versus 28% for those infants who did not receive cooling.

Traumatic brain injury (TBI) remains the leading cause of mortality and disability in the United States. Approximately 1.5 million Americans are affected annually and 5.3 million Americans require assisted living as a result of TBI. TBI can be caused by falls, physical abuse, motor vehicle accidents, sports injuries, and brain trauma experienced while serving in field of battle. TBI is the signature injury of Iraq and Afghanistan conflicts, accounting for approximately 20-25% of the Joint Theater Trauma Registry (JTTR) reviewed combat casualties. Between 2000 and 2015, 559 Service members sustained a moderate/severe/penetrating brain injury. While more TBIs occur in the non-deployed setting (versus deployed), the economic costs of work days lost and long term care for those who have sustained more severe injuries are significant. The US Department of Defense recognizes that gaps remain in the treatment of casualties with moderate to severe TBI at the point of injury and during transport when they are most at risk for secondary brain injury.

Induced hypothermia as a therapeutic treatment for severe TBI has been studied intensively in the last 20 years. Pre-clinical research has consistently demonstrated that therapeutic hypothermia is a promising neuroprotective strategy for treating traumatic brain injury (TBI) by effectively reducing injury-induced increases in intracranial pressure and cellular damage and improving neurological outcomes. However, uncontrolled hypothermia is considered a threat in treating soldiers with TBI engaged in military operations as it causes increased platelet dysfunction and inhibits the coagulation cascade promoting coagulopathy and contributing to the lethal triad. Thus, hypothermia induced by whole-body cooling techniques is contraindicated for combat TBI victims who suffer from multiple wounds.

Post Traumatic Epilepsy (PTE) affects up to 75% of TBI's. PTE is the leading cause of acquired epilepsy in young adult and has proven difficult for patients to manage symptoms. Chronic spontaneous recurrent seizures (CSRS) are common among patients with epilepsy. The mechanisms behind the onset of epileptic seizures after brain injury is not known. A recent study in rats showed that cooling the brain by only 2° C., beginning 3 days after the brain injury and continued for five weeks post-injury, virtually abolished the incidence of post-trauma epileptic seizures.

The American Academy of Sleep Medicine says that chronic insomnia, symptoms that last for at least a month, affect about ten percent of adults. A cooling system that reduces the brain temperature may be an effective treatment for chronic insomnia. A reduction in the metabolism in the brain's frontal cortex occurs while falling asleep and is associated with restorative sleep. Insomnia is associated with increased metabolism in the same region. Cerebral hypothermia is one way to reduce cerebral metabolic activity through frontal cerebral thermal transfer to cool the brain. The results of a recent study showed the effects of thermal transfer to the brain improved sleep latency and sleep efficiency in those with insomnia to patterns similar with healthy adults. The most significant finding from this study is that there is a beneficial impact on the sleep of insomnia patients from the application of a safe non-pharmaceutical mechanism that can be made widely available for home use.

There are an estimated 1.6-3.8 million sports related TBI's that occur in the US annually. Of this amount an estimated 75-80% are mild involving only a brief alteration in consciousness or mental status. A mild traumatic brain injury (mTBI) is commonly referred to as a concussion. Clinically a loss of consciousness for less than 30 minutes, altered mental state for less than 24 hours, or posttraumatic amnesia for less than 24 hours is considered an mTBI. Post concussion syndrome is characterized by experiencing multiple cognitive, somatic, and/or affective symptoms for longer than 3 months. Post concussion symptoms can lead to chronic disability. Repetitive head impacts further complicate the characterization of mTBI by overlapping the emergence and duration of pathogenic events. Chronic repetitive sub concussive head impacts may result in cumulative long term deleterious effects. Common long term health disorders associated with recurrent mTBIs include neurocognitive deficits such as memory loss, attention loss, decreased processing speed, posttraumatic stress syndrome, psychological health problems such as binge drinking, dcprcssion, impairment of social functioning, epilepsy, and altered personality.

There is a need for a selective brain cooling system that can selectively reduce and regulate the brain's temperature. The application of a selective brain cooling system has far reaching implications for first responders, battle field medics, in the emergency department, the surgical suite, the neonatal nursery, during athletic events, and for home use to treat insomnia.

Brain cooling systems that function by circulating cooled water or fluid through channels in a head-worn cap are commercially available. However, these systems are bulky, heavy, challenging to deploy in critical situations, such as on the battlefield and by first responders, and are not practical for most situations. There remains a need for a small, lightweight, battery-powered, universal selective brain cooling system.

### BRIEF DESCRIPTION OF THE DRAWINGS

FIG. 1 illustrates a perspective view of a selective brain cooling system attached to a patient's head.

FIG. 2 illustrates an embodiment of a headliner having multiple bands of segmented cooling units.

FIG. 3 illustrates an embodiment of a headliner band configured as a non-segmented continuous unit that may be cut to adjust the size and length of the band to fit the patient's head.

FIG. 4 illustrates an embodiment of a headliner with multiple layers to enhance heat transfer from the patient's head.

FIG. 5 illustrates an embodiment of a headliner having a single thermally conductive layer that interfaces the patient's head with the cooling unit.

FIG. 6 illustrates a section view of an embodiment of a cooling unit that utilizes a TEC and heatsink.

FIG. 7 illustrates an embodiment of a section view of a cooling unit and heat dissipation unit that utilizes a fan sink.

FIG. 8 shows an embodiment of a headliner coupled to a temperature sensing unit.

FIG. 9 is a schematic depicting an overall temperature control system for a selective brain cooling system.

FIG. 10 illustrates a section view of a neck brace and a headliner attached to a patient.

FIG. 11 shows various possible geometric configurations of a headliner and cooling units placed within different headliner segments.

FIG. 12 shows an embodiment of a headliner having three layers.

FIG. 13 illustrates an embodiment where the neck brace and headliner may be adjusted in different directions or planes.

FIG. 14 illustrates an embodiment of a neck brace having two main components.

### DETAILED DESCRIPTION

A selective brain cooling system is disclosed herein comprising one or more thermoelectric cooling elements (TEC) configured for connecting to a wearable headliner, and/or a neck brace attachable to the head and/or neck of a wearer for selectively reducing and regulating the temperature of the patient's brain, head and/or neck. The system may further comprise one or more of a power controller, electrical wires connecting the headliner to the power controller, electrical wires connecting temperature sensors to the power controller, and a power cord.

One objective of the selective brain cooling system disclosed herein is to protect the brain of the patient from further damage due by cooling all or part of the brain, resulting in a reduction of the intracranial pressure, brain and intracranial temperature, cellular metabolism, and/or cellular damage. This may improve neurological outcomes for patients with various conditions, including traumatic brain injury, mild traumatic brain injury, epilepsy, stroke, birth asphyxia, aneurysms, and other conditions in which cerebral hypothermia is shown to be of therapeutic value.

The selective brain system disclosed herein may reduce the short and long-term effects of neurological impairment due to traumatic brain injury, stroke, epilepsy, cardiac arrest, concussion, birth asphyxia, or other conditions that may result in loss of brain function. The selective brain cooling system disclosed herein may also improve the quality of life for those suffering from insomnia, Alzheimer's disease, and other disorders in which hypothermia is shown to be an effective therapy.

The headliner disclosed herein may provide a surface area configured to provide a greater contact surface area with the patient's head for increased coverage of the head and neck, improved response time for temperature changes, and improved temperature regulation.

The headliner disclosed herein may be designed to interface with one or more thermoelectric cooling elements (TECs) that are oriented with the cold surface of the TEC towards the headliner so that when electricity activates the TECs, the headliner may decrease in temperature. The TEC may also be designed to allow for hot or cold temperatures to be passed to the headliner and on to the patient's head and/or neck.

A heat dissipation unit may be designed to remove heat from the TECs by natural convection, forced convection, heat pipes, phase change materials, and fluid heat exchanger. The heat dissipation unit may interface with the hot side of the TEC that may be oriented away from the head. The heat dissipation unit may remove the heat for immediate dissipation to atmosphere or to be stored in a thermal medium for dissipation at a later time.

The headliner and thermoelectric cooling elements may be organized into modular segments so that the medical personnel attaching the headliner may avoid head trauma sites or provide caregiver access to the head but still be able to provide temperature regulation to the head. The headliner may also be organized into segments that may be modified, re-oriented or removed from the other segments to provide a customizable fit or configuration around head wounds.

The thermoelectric cooling elements may be connected to a power supply and control board to regulate the power supplied to the TECs. The power supplied to the TECs can be used to influence the rate and temperature at which the headliner may alter the temperature of the head and brain. The electronic controller may also provide the ability to regulate the temperature of individual or groups of TECs to provide more specific temperature regulation of a patient's head. The power supply may comprise a portable power supply, such as a battery or generator, or may comprise a transformer configured to accept standard wall outlet electrical power. In some embodiments, the TECs may be powered by wall outlet power but be provided with a battery backup power supply.

The controller may have a battery life indicator that may be a visual or audible signal. The controller may allow the user to set a temperature range and the amount of time the system should take to reach that temperature range. The controller may have the ability to set the length of time for treatment, have visual or audible alarms that signal if the brain temperature goes outside of the set temperature range, and an alarm that signals when the battery life is nearing depletion. The controller may have set programs that are designed to treat commonly encountered scenarios such as but not limited to head wounds. The controller may receive inputs from single or multiple temperature sensors and adjust the power supplied to the cooling units in a closed control loop. The controller may have an alarm that signals when the target brain temperature range has been achieved based on feedback from single or multiple temperature sensors. The controller may also provide automatic shutoff for scenarios that may be dangerous for the patient such as too fast of a temperature drop for any of the organs of the head (eyes, ears, brain, skin, or spine) or a temperature too low for any particular organ. The controller may have alarms for the temperature of the cold side of the TEC or hot side of the TEC moving out of a preprogrammed safe operating range.

The headliner may be made of multiple layers of material where the inner layer directly interfaces with the patient's head and the outer layer interfaces with the thermoelectric cooling elements. The layers may be made of thermally conductive materials to promote transfer of the cooler temperatures from the TECs through the headliner to the patient's head. These layers may be arranged in a series of bands that may have a webbed connection between bands and may be designed to resemble a headliner. The headliner may also be a continuous layer with multiple TECs placed across its surface. The headliner may be a combination of bands that are connected by elastic webbing or straps that allow for distance adjustment or tension adjustment between bands.

The headliner may contain a fluid or phase change material. The headliner may be designed so that the transfer of the cooling effect from the cold side surface of the TEC passes through the fluid or phase change material to reach the inner layer of the headliner. The fluid or phase change material may be used to increase the contact surface area between the TEC and the inner layer of the headliner and consequently increase the contact surface area with the patient's head.

The headliner may be organized into segments and those segments may be adjustable relative to one another. This allows the headliner to be adjustable and allows the headliner to fit heads of different sizes while maintaining maximum contact surface area for optimal cooling. The headliner may utilize adhesive or elastic bands that create tension between the bands or across the headliners surface that may result in compressive forces between the headliner and the patient's head for a secure and adjustable fit. The headliner may also be adjustable through the use of elastic, buckles, clips, friction rings, tension devices, hook and loop tabs, and other such adjustment means.

The selective brain cooling system may be powered by either AC or DC power. The controller may be connected or disconnected from a power supply that may be either a battery pack or wall outlet connection. The controller may also be connected to a mobile power supply such as the power provided in a helicopter, ambulance, or other method of transport. This would allow the patient to have power to the selective brain cooling system while in the field, being transferred to the hospital, transferred within the hospital, or with a loss of power to the hospital.

The selective brain cooling system may be a deployable device that can effectively induce cerebral hypothermia in a therapeutically beneficial time frame. The time to target temperature and the temperature range of the treatment may vary based on the desired application.

The selective brain cooling system may be used for as long as is deemed necessary by the administering doctor or physician, and the temperature of the cooling system may be adjusted during use to increase or decrease the temperature and duration of cooling to the brain.

The selective brain cooling system may be a two-component system that includes a brain cooling system partnered with a cooling neck brace designed to interface with the brain cooling system. The cooling neck brace may be designed to cool the neck and back of the head while providing support and stability to the head and neck. The neck brace may comprise structures configured to support the neck, or may simply comprise a flexible neck wrap that provides little or no neck or head support, or patches or bands configured for disposition on or about the neck.

The selective brain cooling system and neck brace may be designed to be a single patient use system where the components that interface with the patient are discarded after use and the power supply and controller may be used with multiple patients. The headliner and all attached TEC and heat dissipation units may be discarded. The connection between the controller and headliner will be detachable, allowing for the controller and power supply to be reused with a new headliner system. The temperature sensing system may be reusable if sanitized between patients. Temperature sensors such as a microwave emission sensor, tympanic membrane probe, or skin temperature may be sterilized and reused. Invasive and/or non-invasive temperature sensors may be used to record brain temperature and core temperature. The headliner may be reused in situations where the headliner is not contaminated beyond acceptable sanitization protocols.

The selective brain cooling system may make use of multiple methods for measuring skin, optical, tympanic, and brain temperature. The temperature sensing unit may be attached to the headliner or a separate unit that is used independently of the headliner. Temperature sensors such as skin or microwave emission are topical systems that can be applied before, during, or after applying the headliner to the patient. The electronic controller may make use of a thermal mapping model to analyze temperature readings from multiple sensors and make temperature regulation adjustments to effectively regulate a patient's head, neck, and brain temperature in an optimal manner. The controller may have the option thr a clinician to set maximum and minimum temperatures for skin, ear, eye, and brain. The controller would adjust the temperature of the cooling system based on the clinician's inputs and feedback from the temperature sensing unit.

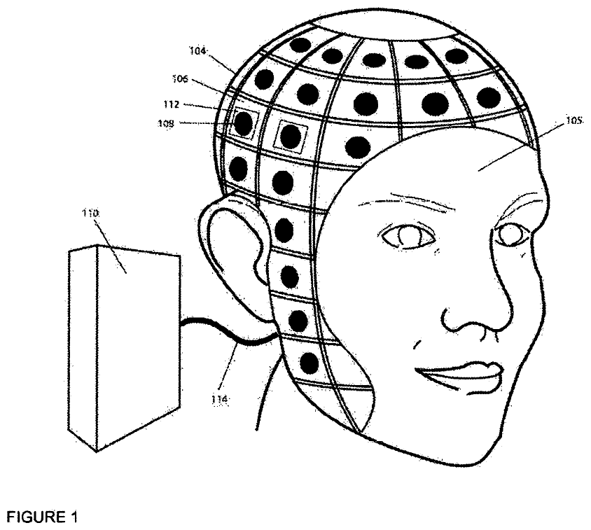

The embodiment of FIG. 1 shows a perspective view of the selective brain cooling system 100 attached to a patient head 105. As may be seen in the embodiment of FIG. 1, the headliner 102 wraps around the patient's head in multiple bands 104 of segmented blocks 106. Within each band are multiple blocks. Each block contains at least one cooling unit 108. The cooling system is connected to the power control unit 110 and the heat dissipation unit 112. The power control unit may include a controller and a power supply. The power supply may be either a battery pack, fuel cell, or a power cable connected to a wall outlet. The power control unit is connected to the cooling system via power cables 114. The power cables may vary in number and size. In this embodiment, there are multiple power cables running from the controller to the headliner. There may be a single power cable that runs from the controller to the headliner and neck brace, or a power cable for the headliner and a power cable for the neck brace. The single power cable may contain multiple power lines to supply power to different bands or segments on the headliner. The single power cable for the neck brace may also contain multiple power lines inside a single power cable connecting the neck brace and controller. The power control unit may receive power from ether a AC or DC power source. The power supply may provide power to the controller that may provide power to the headliner and neck brace. The controller may receive feedback from a temperature sensing unit that is not shown.

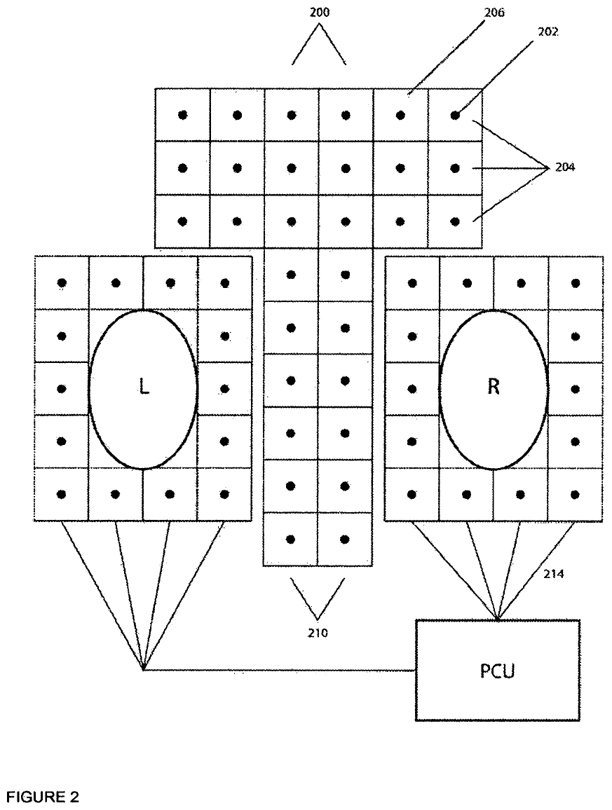

FIG. 2 illustrates an embodiment of the headliner 200 as laid flat, and cooling unit 202. The headliner may include multiple bands 204 and each band may be divided into one or more segments 206 that contains a cooling unit. The bands may be connected together 210. One portion of the headliner may be positioned at the rear of the patient's head. Another portion may be positioned at the top of a patient's head. Left (L) and right (R) side portions may be positioned at the sides of the patient's head. The side portions may be provided in any suitable shape, such as round, U-shaped, square, curved or polygonal. The openings (L) and (R) may be disposed over the patient's left and right ears, respectively. Other portions (not shown) may be configured for disposition over all or part of a patient's face, such as on a forehead, around the eyes and/or nose. The portions may be provided in a flat sheet that may be separated as desired for coverage of the head, or may be provided in the form of a cap from which various portions (or bands or segments thereof) may be removed or re-oriented.

Each band may be flexible and may conform to the patient's head independently of the other bands. Each band may be subdivided and segments may be removed (such as by cutting or tearing the block from the headliner), or repositioned so as to permit ready access to the head, such as if a head wound requires access for treatment. Such configuration may permit application of the headliner on or about a head suffering from swelling, laceration or other disfigurement. In other embodiments, the headliner may comprise a frame that may disposed TEC's about the head in a manner than reduces pressure on certain portions of the head and accommodates swelling and changes to the shape of the head. Each cooling unit may be powered by its own power wire 212 that may be grouped together into a power cable 216. This can allow for the removal of bands or strips of headliner, or of segments without having to remove an entire band. In other embodiments, the segments may be mounted to a band in such a way as to allow re-orientation of the segments along the band, such as by sliding along the band, or by removal and re-mounting, such as by hook and loop fastener.

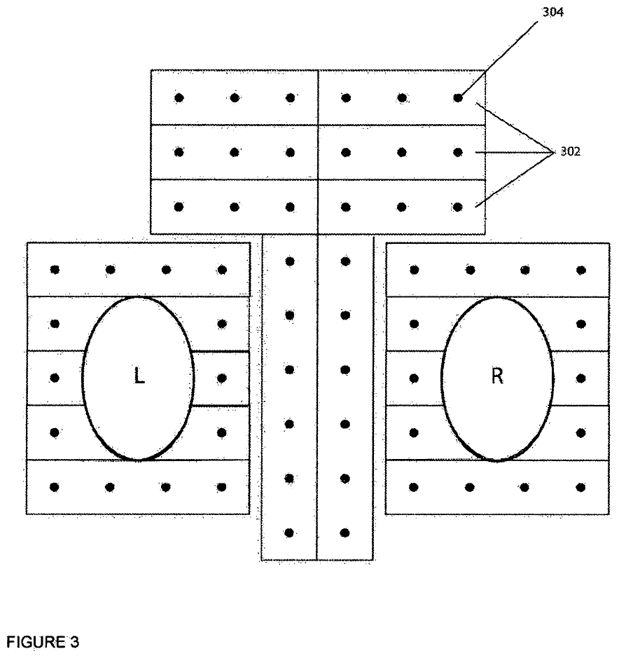

FIG. 3 illustrates an embodiment where the headliner band 300 is made of continuous bands 302 with multiple cooling units 304 positioned along its length. The headliner band may be made of a fabric or polymeric material that allows for removal of a cooling unit or group of cooling units. The removal of a cooling unit may be accomplished by cutting, tearing, or other means of removing portions of the headliner band to adjust size and length of the band. This would provide the ability to adjust the size of the band to fit the patient's head without using discrete segmented sections.

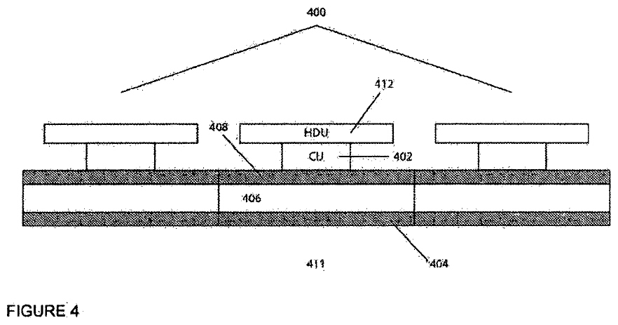

FIG. 4 illustrates an embodiment of the headliner 400 and cooling unit 402. The headliner may be made of multiple layers. In this embodiment, the headliner is made of an inner layer 404 that interfaces with the patient's head 411 and the middle layer 406 that is made of a high thermal conductivity material. This high thermal conductivity material may be made of an effective cold sink material such as but not limited to phase change material, a highly conductive fluid, or a thermally-enhanced polymeric or fabric layer. The middle layer interfaces with the inner layer and the outer layer 408. The inner and outer layers may be made from the same fabric or polymeric material. The outer layer also interfaces with the cooling unit. The cooling unit interfaces with the heat dissipation unit 412.

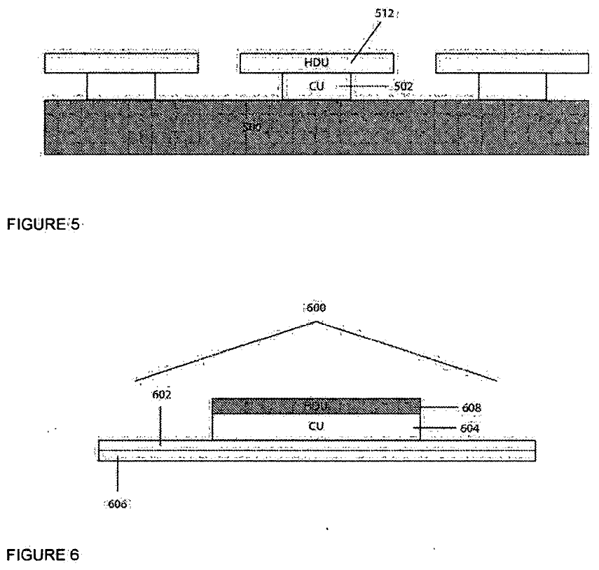

FIG. 5 illustrates an embodiment where the headliner 500 is made of a single thermally conductive layer. This layer may be made of fabric or a polymeric material. The headliner may be designed to enhance the heat transfer between the cooling unit 502 and the patient's head by use of a doped polymeric material, integrated metallic powders, integrated metallic wires, ceramic flakes or powders, and the combination thereof. The headliner interfaces with the patient's head and the cooling unit. The cooling unit may interface with a heat dissipation unit 512 to dissipate heat to atmosphere.

FIG. 6 illustrates an embodiment of the cooling unit 600. The cooling unit may include a thermally conductive medium 602 interface between the thermoelectric cooling element (TEC) 604 and the headliner 606. The thermally conductive medium may be a thermal grease, thermal epoxy, or other non-fluid and non-gas commercially available thermal interface material. The TEC also interfaces with the heat dissipation unit 608. In this embodiment the heat dissipation unit is a heatsink. The heatsink may be made of a metallic, ceramic, or other appropriate thermally-conductive material and be designed to dissipate heat under natural or forced convection circumstances.

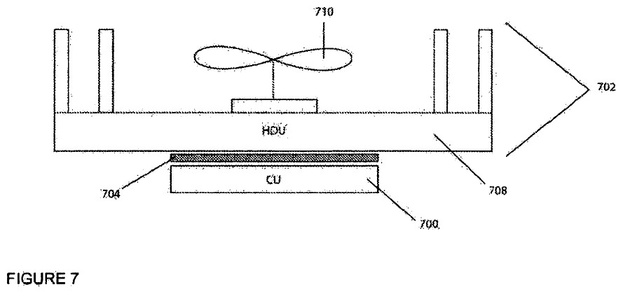

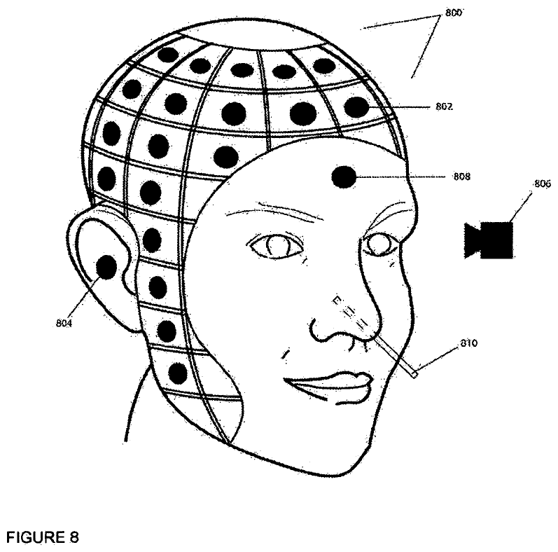

FIG. 7 illustrates an embodiment of the thermoelectric cooling element 700 and heat dissipation unit 702. The heat dissipation unit in this embodiment may include a thermally conductive medium 704 that interfaces the heat dissipation unit and the cooling unit. The thermally conductive medium may be a thermal epoxy, thermal grease, or other thermally conductive heat transfer component designed to enhance the heat transfer from one object to another. The heat dissipation unit makes use of a fan and heatsink combination where a heatsink 708 and electric fan 710 are partnered together to increase the heat dissipation to atmosphere.

FIG. 8 illustrates an embodiment of the headliner 800 with multiple attached temperature sensing devices. The temperature sensing unit may use technology that allows for temperature sensing of the brain. The temperature sensing unit may also use technology that allows for temperature sensing of the surface of the patient's head and uses a computational model to determine the temperature of the brain. The skin temperature may be sensed in multiple locations across the head by a skin temperature sensor 802. There may also be an optical temperature sensor 806, and a sensor that detects microwave emissions from the brain 808. The optical sensor may be a brain tunnel temperature sensor designed to look at the inner corner of the eye or any other optical eye sensor that can report an accurate temperature of either the brain or the eye. A nasal passage temperature sensor 810 and/or tympanic temperature sensor 804 may be utilized by the cooling system. A combination of these sensors may be used in conjunction with the electronic controller to asses and regulate the temperature of a patient's head, neck, and brain.

In yet other embodiments, the headliner may be provided as one or more patches, with each patch comprising one or more TECs. The patches may be positioned about a head as may be required for selective cooling. The patches may be held against the head by adhesive, or may be taped to the head, or may be placed under bands or within a head covering. In some embodiments, the head may be shaved prior to application of the headliner.

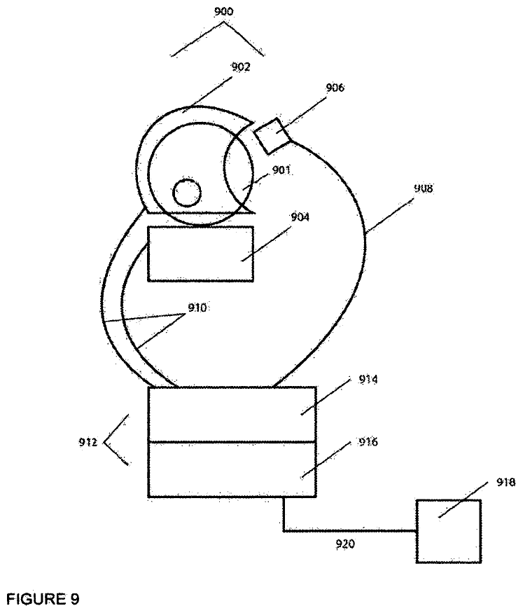

FIG. 9 is a schematic depicting an overall temperature control system for the selective brain cooling system 900. The headliner 902 fits over the patient's head 901 and interfaces with the power control unit 912 by power cables 910. The power control unit is broken into the power supply 916 and the controller 914. The controller connects to the headliner, neck brace 904, the temperature sensing unit 906 via data cables 908, and the power supply. The power supply connects to a power source 918 via a power supply cable 920. The temperature sensing unit interfaces with the patients head to provide information to the controller. The controller adjusts the power sent to the neck brace and headliner based on modeling data and the input from the temperature sensing unit. The controller is powered by the power supply which may receive its power from a battery pack or either an AC or DC source. Thus, one or more of the TECs may be selectively activated so as to provide cooling to all or part of the patient's head.

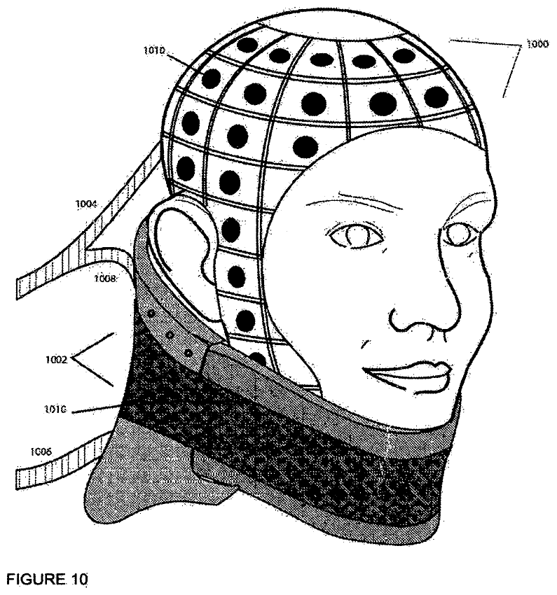

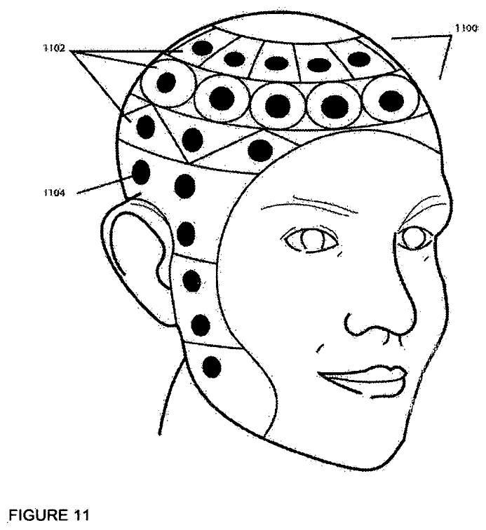

The example system in FIG. 10 includes an adjustable single or multiple piece head covering element being referred to as the headliner. FIG. 10 illustrates an example of placement of cooling units around the head, shows a gel layer placed between the head and the headliner, shows cooling units along the neck brace, shows power cables, and shows ear holes. The headliner may also be designed to interface with a neck brace to stabilize the head and neck. FIG. 10 shows an embodiment of a section view of the headliner 1000 and the neck brace 1002. This embodiment shows multiple cooling units placed around the patient's head and neck. The headliner may be designed to fit around a patient's head leaving the ears and face accessible and the neck brace designed to stabilize a patient head and neck. There may be a thermally conductive gel (not shown) applied to the head and neck to promote heat transfer between the headliner and head. The neck brace may be designed to contain the same cooling units 1010 that the headliner is designed to use. The headliner power cable 1004 may be a stand alone power cable and the neck brace power cable 1006 may also be stand alone. The headliner power cable and the neck brace power cable may join together into a single headliner/neck brace power cable 1008. All of these components may be designed to fit cooperatively together, be lightweight, and be provided as a portable system. The selective brain cooling system may be carried, applied, and operated by a single person.

FIG. 11 illustrates an embodiment of the headliner 1100 where the segments 1102 may have various geometric shapes configured to conform to the head in different manners so as to assist in selective application of cooling. The inner layer, middle layer, and outer layers may have the same geometric shape or all three may be different. Thermoelectric coolers come in different configurations such as donut shaped, square, rectangular, circular, and a three dimensional pyramid. The headliner may take on an optimal shape to allow for the highest accuracy and rate of temperature regulation to the brain. The cooling units 1104 may rest inside the various headliner segments. The segments may be physically separated into patches or bands that may be applied to the head.

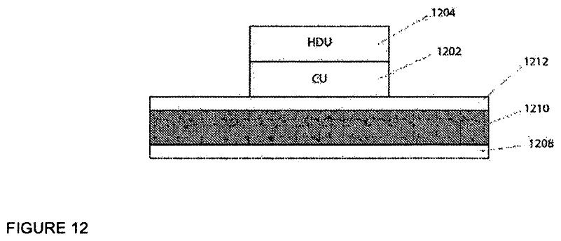

The headliner 1200 may contain a middle layer 1210 that contains a material of significantly higher thermal conductivity than the inner and outer layers, as may be seen in FIG. 12. The inner layer faces the patient head and the outer layer faces the cooling unit. The size and thickness of the three layers may vary. The inner layer 1208 may be designed to interface with the patient's head and the outer layer 1212 may be designed to interface with a source of heat transfer such as a TEC 1202. The TEC may interface with a heat dissipation unit 1204 for transferring heat to atmosphere. The middle layer may contain a fluid or phase change material, and be sealed inside the inner and outer layer, as well as sealed into segments. The headliner may make use of separate sealed sections of the middle layer, and each separate section would utilize a cooling unit. This would allow the sealed section to be cut or removed from one another without damaging the neighboring segments. In the event of a head wound or should direct access to the brain for monitoring intracranial pressure be required, a clinician could remove the segments that would impair treatment of a wound or direct access to the brain, but still be able to apply and use the selective brain cooling system. In order to make use of these separate sealed segments the headliner may be constructed by any one of the conventional bonding techniques such as but not limited to: ultrasonic welding, vibration welding, heat welding, radio frequency welding, electromagnetic welding, induction welding, thermal sealing, adhesive bonding techniques, or injecting the thermal material after the construction of the headliner is complete where the headliner is made of a self-sealing material.

The headliner and neck brace may be made from polymeric materials such as blow molded plastics, nylon, fiberglass, rubber, metal, and any combination thereof. The material is able to withstand contraction from rapid decrease in temperature and subsequent expansion upon warming without sustaining short or long term damage. The inner layer of the headliner is thin enough to conduct the cooler temperatures at the surface of the TEC to the patient's head and neck. The temperature transfer is done at such a rate that is sufficient to quickly slow brain metabolism and inhibit potential neurological damage. The outer layer of the headliner may be made of the same or similar material and have the same properties as the inner layer. The headliner and neck brace may be rigid enough to support the patient's head without deforming when being worn. The headliner may also be soft and cloth like with more rigid sections to support the head and neck. The headliner and neck brace may also be made of or have a layer of sponge or foam like material. This foam material would be supportive to the head and neck but thin enough to provide sufficient heat transfer to the patient's head. The foam material may be surrounded or supported in areas by a more rigid material that supports the head and neck.

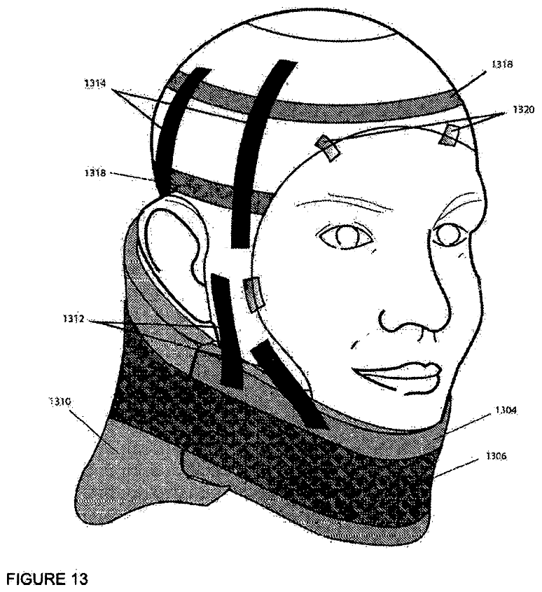

FIG. 13 shows the selective cooling system 1300 applied to a patient's head and neck. The tension of the headliner may be adjusted as well as the tension between the neck brace and headliner. The headliner 1302 and neck brace 1305 may be placed in tension by adjusting head-neck straps 1312 that are designed to anchor to both the headliner and neck brace. The neck brace may be adjustable around the patient neck by adjusting a neck brace strap 1306 around the neck brace front 1304 and securing it to the neck brace rear 1310. The headliner may be adjusted to fit a patient's head by adjusting the vertical straps 1314 and the horizontal straps 1318. The headliner may also be secured to the patient's neck or face by the use of tape 1320. The headliner is of a universal size to insure conformity to all head sizes. The headliner may be made of multiple components that can be adjusted relative to one another to provide a secure fit to the patient's head. The components may be adjusted in multiple planes or directions. The headliner may also be made of an elastic or stretchable material that is stretched over the patient's head. The headliner may be made of an elastic material or make use of elastic and adhesive bands that are used to adjust the size of the headliner to fit the patient. The headliner and neck brace may include flexible elastic straps that provide tension between two points on the headliner or neck brace. The tension provided by multiple straps may provide compression between the headliner and the patient's head. The tension between the elastic straps or bands may provide size adjustments on the headliner and between the headliner and neck brace. The tension bands are elastic enough to provide for adjustment yet resilient enough to maintain compression to the patient's head during use. The tension bands may also be of a material that will not have decreasing mechanical properties over the course of use of the selective brain cooling system. The tension bands may make use of anchors or mounts that provide a secure attachment to the headliner or neck mace as well as secondary anchor sites. The secondary anchor sites may be variable in size and location to provide for a custom fit to the patient's head. The anchor sites may be designed to receive and maintain connection with the tension bands/straps. Other alternative adjustment mechanisms include but are not limited to straps, bands, tape, belts, hook-and-loop fasteners, ratchets with straps, zip ties, and other attachment devices.

The selective brain cooling system is designed in some embodiments to be used as a headliner and neck brace combination, but the two components can be used independently of one another. The neck brace and headliner can be used as standalone systems to allow maximum versatility for application in unique circumstances that would not allow for a neck brace or a medical device to be attached to the head. The selective brain cooling system and neck brace are designed to be portable and suitable for field use, in ambulances and first responders, athletic fields, aircraft, marine vehicles, spacecraft, emergency care centers, hospitals, home use, and work sites.

The headliner may be made of bands or segments that are joined together to mimic the shape of a patient's head. The headliner may be made of elastomeric materials to allow for flexibility and easy storage. The headliner may be foldable or packable for easy transport inside a carrying case. The headliner bands may be connected by an elastomeric material to allow for stretching and flexibility between bands while adjusting the headliner to the patient's head. The multiple bands provide sufficient coverage of the patient's head to allow for temperature regulation of the brain and also allow for quick and easy donning of the headliner. The headliner may make use of flexible adjustment mechanisms between the headliner bands in multiple locations across the headliner. This would provide for adjusting the size of the headliner in multiple planes as well as being able to pull the headliner securely down onto the patient's head by attaching it to the neck brace. FIG. 13 shows an embodiment of the headliner and neck brace using various adjustment mechanisms in multiple directions. The number of adjustments and means of adjustment may vary.

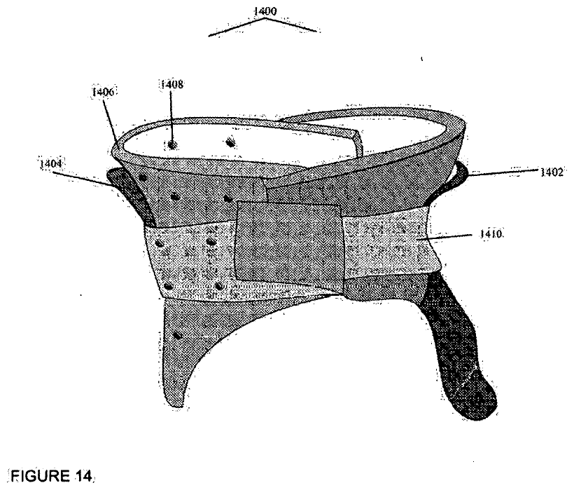

FIG. 14 shows an embodiment of the neck brace 1400 that uses a two part system of a front brace 1402 and a rear brace 1404 with an adjustable strap 1410 and cooling units 1408 installed on the rear section of the neck brace. This illustration shows an embodiment of the neck brace where there is a foam layer, hard surface for stabilization, cooling units, a single power cable, adjustable straps, and an opening for the head and neck. The neck brace may use foam 1406 or other material commonly used in neck braces as the interfacing surface between the front and rear braces and the neck. The neck brace supports the head and neck of a patient. It has a curved upper portion to accommodate the natural curvature of the cervical spine. The cm veil portion may be designed to hyperextend and position the neck upwards, which allows for easy access to the large neck blood vessels should a catheter be required or if resuscitation is needed. The position of the head and neck allows for paramedics or first responders to treat a patient in a stable yet accessible position. The neck brace may be made of a polymeric material that is capable of withstanding the rapid contraction from cooling and the rapid expansion upon warming without sustaining any irreversible damage. The neck brace may be made from the same means as the headliner which include but are not limited to those noted above. The neck brace may be designed to allow for the same or similar cooling units attached to the headliner to be attached to the neck brace in such a way as to provide temperature regulation to the neck and rear of the head. The neck brace should be supportive enough to support the head and neck without deforming under the patient's weight, but still be designed to allow for the temperature regulation of the neck and head to sufficiently slow brain metabolism, reduce intracranial pressure and temperature, and prevent or reduce neurological damage.

Padding that is not shown on these drawings may be included on the headliner and neck brace. This padding may provide a comfortable and cushioning effect between the headliner to patient's head and neck brace to patient's head and neck. The padding may be supportive enough to stabilize the patient's head and neck for transport and be designed to absorb small forces transmitted during transport. The padding may be designed to enhance the heat transfer from the cooling units by being of a material with a high thermal conductivity. The padding may increase the contact surface area between the neck brace and neck and head. This increased contact surface area may allow for improved temperature regulation of the head and neck.

The selective brain cooling system and the two components of the headliner and neck brace are two devices that can be used independent of one another in most environments and circumstances. The cooling system is compact enough to have ready in an emergency response vehicle or on the field of combat for quick application to a patient. The cooling system is also accurate enough to be adequately used in the clinical setting. During a surgical procedure the brain cooling system can reduce the brain's temperature to slow cellular metabolism and allow for extended resuscitation attempts during cardiac arrest or respiratory arrest. The selective brain cooling system may also slow cellular metabolism and cell death in those suffering from stroke, cerebrovascular trauma, traumatic brain injury, and other situations that affect the brain's integrity. The operation of the selective brain cooling system is simple enough to be applied in the field quickly and yet accurate enough to be used in many other therapeutic enviimments. The universal means of application and adjustments to the temperature regulation settings enables clinicians and first responders to protect a patient from severe and irreversible neurological damage.

The headliner middle layer may contain a high thermal conductivity material. This material may be a solid, liquid, gas, or combination thereof. The preferred thermal conductivity material would be able to withstand sudden decreases and increases in temperature without significant changes in the composition or material properties of the material. A combination of multiple high thermal conductivity materials may be used. Additives may be included in the inner, middle, and outer layers of the headliner in order to promote heat transfer. Additives may also be used to prevent any bacteria from growing on the surface of the headliner. A stable fluid material is R-134A. This fluid is environmentally safe, nonflammable, no known reproductive toxins, non-irritating with skin contact, freezing point below −150° F., and is considered a stable fluid at low temperatures. An organic, inorganic, or composite phase change material may be used in place of a fluid. A phase change material (PCM) is a substance that exhibits a large latent heat of fusion. During a phase transition the PCM is capable of absorbing or releasing large amounts of thermal energy. When PCMs reach the temperature at which they change phase, the PCM may absorb a large amount of heat while maintaining a relatively constant temperature. A phase change material can be created in a small bead form and suspended in a fluid. A thermal grease may be used as the middle layer of the headliner. A thermal grease is a high thermal conductivity but electrically insulating compound that may comprise a polymerizable liquid matrix and a filler material. Typical matrix materials are epoxies, silicones, and urethanes. Common filler materials are aluminum oxide, boron nitride, zinc oxide, silver, gold, or copper powder flakes, and aluminum nitride.

The inner layer of the headliner or the patient's head may be coated with a high thermal conductivity material to enhance the heat transfer between the headliner and head. A low viscosity liquid or a gel material may be applied directly to the head or to the inner layer of the headliner. This material would be compressed between the headliner and head during application of the cooling system and force air pockets out of the interface between the headliner and head.

The number and size of the thermoelectric cooling elements and the corresponding heat dissipation units may vary. The headliner may have many cooling units that correlate to smaller headliner segments and thinner headliner bands. The more headliner bands and segments the more adjustable the headliner becomes, but there may be a limit to the number of cooling units that can be placed on the headliner. Cost may limit the number of cooling units, or research may show that a smaller number of cooling units will suffice. A single large TEC may be used to cool the headliner and be able to cool the brain sufficiently, or a moderate number of middle sized TECS may be placed around the head and neck, and a large number of very small TECs may be placed in many locations on the headliner.

Measurements of body temperature may not reflect brain temperature during or after an ischemic event. A temperature sensor placed in the temporal muscle may not show the actual temperature of the deep brain because of the superficial extracranial position of the sensor. The tympanic membrane temperature reflects the whole brain temperature closely. A tympanal thermocouple probe may closely reflect the deep brain temperature and may provide a non-invasive means for brain temperature monitoring. The temperature sensing unit of the selective brain cooling system may utilize a tympanic temperature sensor.

The selective brain cooling system may utilize an optical sensor designed to report core body temperature by observing the inner corner of the eye. The human brain and core temperature can both be noninvasively and continuously measured using the Abreu BTT 700 made by Brain Tunnelgenix Technologies. The brain temperature is measured by observing the thermal storage area in the brain and an area of the skin located at the corner of the eye. Thermal energy is transferred from the eye to the sensor without obstruction. The Abreu system also uses a reusable pen like probe for taking spot measurements on any skin surface. The temperature sensing system may use a system such as the Abreu in combination with other temperature sensors and temperature sensing modalities.

Skin temperature at the head, neck, and face may be measured by the temperature sensing unit. The type and location of the temperature sensor may vary. Temperature sensors that may be used but are not limited to are; thermal or infrared imaging sensors, thermistors, RTDs, thermocouples, or any combination thereof. The temperature sensors may be designed to be integrated into the headliner and neck brace or be applied independent of the cooling system. The skin temperature sensor may be applied directly to the skin at the scalp, face, neck, and body.

The temperature sensing unit may use a non-invasive microwave emissions sensor. The temperature sensor may be designed to rest on the forehead and measure the microwaves that pass unimpeded though the skull. As temperature increases so do the microwave emissions. The sensor would measure the microwave emissions and calculate the temperature 1.5 centimeters beneath the skull. The microwave sensor would provide the temperature sensing unit with a brain temperature that the controller would use as feedback during the cooling systems operation.

Core temperature may be measured through the use of a rectal sensor, a sensor that is ingested and wirelessly transmits the core temperature, through tympanic temperature, or nasal passage temperature.

Selective brain cooling is defined as lowering the average brain temperature below that of arterial blood. Cooling of the carotid arteries by cooling the neck may influence the temperature of arterial blood entering the brain.

Thus, the brain cooling system may include a variety of configurations, such as those described in the following numbered clauses:

1. A selective brain cooling system comprising a headliner configured for disposition on a head; and two or more first thermoelectric cooling elements (TECs) mounted to the headliner.

2. The system of clause 1, the headliner comprising two or more segments, each segment having at least one of the first TECs.

3. The system of clause 1, the headliner comprising two or more bands, each band comprising two or more segments, each segment having at least one of the first TECs.

4. The system of clause 1, the headliner comprising a plurality of patches, each patch having at least one of the first TECs.

5. The system of clause 1 further comprising a neck brace configured for disposition on a neck; and two or more second TECs mounted to the neck brace.

6. The system of clause 1 further comprising a power supply electrically coupled to the first TECs so as to provide electrical power thereto; and a power controller electrically coupled between the power supply and the first TECs to control the electrical power provided by the power supply to the first TECs.

7. The system of clause 6 further comprising a temperature sensor configured to sense the temperature of the head or a portion thereof.

8. The system of clause 7, wherein the temperature sensor provides temperature data to the power controller, the power controller being configured to control the electrical power in response to the temperature data.

9. The system of clause 8, the temperature sensor being separate from the headliner.

10. The system of clause 8, the headliner further comprising the temperature sensor.

11. The system of clause 6, the power supply comprising a battery, or wall outlet electrical power, or both.

12. The system of clause 1, the headliner being a cap configured to cover at least part of the head.

13. The system of clause 12, the headliner comprising removable segments.

14. The system of clause 12, the headliner comprising removable bands.

15. The system of clause 1, the headliner being configured to cover all or part of the scalp.

16. The system of clause 1, the headliner being configured to cover all or part of the face.

17. The system of clause 1, the headliner being configured to cover all or part of the scalp and the face.

18. The system of clause 1, the headliner comprising two or more portions, each portion being configured to cover a part of the head.

19. The system of clause 18, a first portion configured to cover the back of the head, a second portion configured to cover the top of the head, a third portion configured to cover a first side of the head, and a fourth portion configured to cover a second side of the head.

20. The system of clause 19, the third portion and the fourth portion each being configured to fit about an ear.

21. The system of clause 1, the headliner being configured to allow physical removal of at least one of the first TECs.

22. The system of clause 1, the headliner being configured to allow physical re-orientation of at least one of the first TECs.

23. The system of clause 1, each of the first TECs being mounted to the headliner so as to place the cold side of the TEC toward the head and the hot side of the TEC away from the head.

24. The system of clause 23, further comprising two or more heat sinks, each heat sink being thermally coupled to at least one of the first TECs.

25. The system of clause 23, the headliner comprising a thermally-conductive layer, the cold side of each of the first TECs being disposed against the thermally-conductive layer.

26. The system of clause 1, the headliner configured for adhesive disposition on the head.

27. The system of clause 1, the headliner configured for compressive disposition on a head.

28. The system of clause 1, the headliner comprising a frame configured to allow placement of one or more of the first TECs on the head so as to minimize pressure to the head.

29. A selective brain cooling system comprising a neck brace configured for disposition on a neck; and two or more thermoelectric cooling elements (TECs) mounted to the neck brace.

30. The system of clause 29, the neck brace comprising a flexible wrap configured to cover all or part of the neck.

31. The system of clause 29, the neck brace comprising a structure configured to support the neck and head.

32. The system of clause 29, the neck brace comprising two or more segments, each segment having at least one of the TECs.

33. The system of clause 29, the neck brace comprising two or more bands, each band comprising two or more segments, each segment having at least one of the TECs.

34. The system of clause 29, the neck brace comprising a plurality of patches, each patch having at least one of the TECs.

35. The system of clause 29 further comprising a power supply electrically coupled to the TECs so as to provide electrical power thereto; and a power controller electrically coupled between the power supply and the TECs to control the electrical power provided by the power supply to the TECs.

36. The system of clause 35 further comprising a temperature sensor configured to sense the temperature of the neck or a portion thereof.

37. The system of clause 36, wherein the temperature sensor provides temperature data to the power controller, the power controller being configured to control the electrical power in response to the temperature data.

38. The system of clause 37, the temperature sensor being separate from the neck brace.

39. The system of clause 37, the headliner further comprising the temperature sensor.

40. The system of clause 35, the power supply comprising a battery, or wall outlet electrical power, or both.

41. The system of clause 29, the neck brace being configured to cover at least part of the neck.

42. The system of clause 41, the neck brace comprising removable segments.

43. The system of clause 41, the neck brace comprising removable bands.

44. The system of clause 29, the neck brace comprising two or more portions, each portion being configured to cover a part of the neck.

45. The system of clause 44, a first portion configured to cover the back of the neck, a second portion configured to cover the front of the neck.

46. The system of clause 44, a first portion configured to cover a first carotid artery of the neck, a second portion configured to cover a second carotid artery of the neck.

47. The system of clause 29, the neck brace being configured to allow physical removal of at least one of the TECs.

48. The system of clause 29, the headliner being configured to allow physical re-orientation of at least one of the TECs.

49. The system of clause 29, each of the first TECs being mounted to the neck brace so as to place the cold side of the TEC toward the neck and the hot side of the TEC away from the neck.

50. The system of clause 49, further comprising two or more heat sinks, each heat sink being thermally coupled to at least one of the TECs.

51. The system of clause 49, the neck brace comprising a thermally-conductive layer, the cold side of each of the TECs being disposed against the thermally-conductive layer.

52. The system of clause 29, the neck brace configured for adhesive disposition on the neck.

53. The system of clause 29, the neck brace configured for compressive disposition on a neck.

54. The system of clause 29, the neck brace comprising a frame configured to support the head and neck, and to allow placement of one or more of the TECs on the neck so as to minimize pressure to the neck.

55. A method of selectively cooling a brain, the method comprising disposing a headliner on or about a head, the headliner comprising two or more thermoelectric cooling elements (TECs); and electrically activating at least one of the TECs.

56. The method of clause 55, further comprising controlling the electrical activation of the TECs with a power controller.

57. The method of clause 55, further comprising configuring the headliner for disposition over one or more parts of the head.

58. The method of clause 57, wherein the headliner comprises one or more segments, each segment including at least one of the TECs, and the configuring comprises removing a segment from the headliner.

59. The method of clause 57, wherein the headliner comprises one or more bands, each band including at least two of the TECs, and the configuring comprises removing a band from the headliner.

60. A method of selectively cooling a brain, the method comprising disposing a neck brace on or about a neck, the headliner comprising two or more thermoelectric cooling elements (TECs); and electrically activating at least one of the TECs.

61. The method of clause 60, further comprising controlling the electrical activation of the TECs with a power controller.

62. The method of clause 60, further comprising configuring the neck brace for disposition over one or more parts of the neck.

63. The method of clause 62, further comprising disposing the neck brace over at least one of the carotid arteries.

64. The method of clause 62, wherein the neck brace comprises one or more segments, each segment including at least one of the TECs, and the configuring comprises removing a segment from the neck brace.

65. The method of clause 62, wherein the neck brace comprises one or more bands, each band including at least two of the TECs, and the configuring comprises removing a band from the neck brace.

66. A headliner comprising one or more thermoelectric cooling elements.

67. A neck brace comprising one or more thermoelectric cooling elements.

68. A portable selective brain cooling system for controlling the temperature of a patient's brain and brain stem by controlling head and neck temperature, the system comprising a source of heat transfer; and a heat transfer material; and a headliner; and a temperature sensing system; and a heat dissipation system; and a neck brace; and a power control system, said headliner having flexible and adjustable segments being of a size to fit around the head of the patient leaving the face and ears exposed, said headliner being flexible and configurable to be folded to a smaller size with a tiat orientation, and a secondary orientation where the headliner is adjustable to be placed downward or around the head in a manner that allows for the majority of the head to be covered by the headliner except for the face and ears, said headliner may have a fluid, phase change material, or high thermal conductivity material inside the headliner segments, where the source of heat transfer makes contact with the high thermal conductivity material and said material is in contact with the inner surface of the headliner that will make contact with the surface of the patient's head, wherein said heat transfer material has a different temperature than the head of the patient and is arranged to be in contact with the source of heat transfer and the inner surface of the headliner such that heat is transferred from the patient's heat to the heat transfer material and to the heat transfer source.

69. The system of clause 68 wherein said heat transfer material interfacing with said heat transfer source is selected to be cooler than the temperature of the head of the patient when the heat transfer source is activated by the power control system, the power control and temperature control systems being configured to be attached to or located within the headliner.

70. The system of clause 69 wherein said heat transfer source may comprise of a single or multiple thermoelectric cooling elements (TEC), said TEC being part of a cooling assembly designed to interface with the headliner and the heat transfer material inside the headliner, said TEC being oriented so that the cold side of the TEC is oriented towards the heat transfer material and the hot side of the TEC is oriented away from the patient's head, said TEC being interfaced with a heat dissipation system that dissipates heat from the TEC to atmosphere.

71. The system of clause 68 where said headliner includes either adhesive or elastic straps attached to the outward facing surface of the headliner, overlying multiple segments of the headliner, adapted such that the adhesive or elastic straps can be used to tighten or loosen the fit of the headliner on the patient's head to adjust contact surface area between the headliner and the patient's head and provide a secure interface.

72. The system of clause 71 wherein said headliner includes at least three layers, an inner layer that makes contact with the patient's head and the thermally conductive middle layer, the thermally conductive middle layer that makes contact with the inner layer and outer layers of the headliner, the outer layer that interfaces with the middle layer and the source of heat transfer, the source of heat transfer that interfaces with the outer layer and the heat dissipation system, where the heat dissipation system interfaces with the atmosphere.

73. The system of clause 72 wherein said headliner inner and outer layers may be made of a polymeric material that is selected from the group of silicone, urethane, silicone-polyurethane block copolymer, and a thermoplastic elastomer.

74. The system of clause 72 wherein said headliner middle layer may be made of a thermally conductive material such as phase change material, thermally conductive fluids, or a thermally enhanced polymeric material.

75. The system of clause 70 wherein said headliner includes a single layer where the inner surface interfaces with the patient's head and the outer layer interfaces with the heat transfer source.

76. The system of clause 75 wherein said headliner may be made of a polymeric material selected from the group of silicone, urethane, silicone-polyurethane block copolymer, and a thermoplastic elastomer.

77. The system of clause 76 wherein the polymeric material may be made to enhance the thermal conductivity thereof, via a highly thermally conductive additive dispersed within the polymeric material.

78. The system of clause 75 wherein the headliner may be made of a fabric material that is modified to enhance the thermal conductivity thereof through the use of one or more mechanisms selected from the group of conductive coatings, conductive filler doping, phase change materials, knit-in or wound wires, thermally conductive powders, and combinations thereof.

79. The system of clause 70 wherein the headliner may be made of an inner layer that interfaces with the head and the source of heat transfer such as a TEC, where the TEC cold side is oriented towards the patient's head and makes contact with the inner layer of the headliner, where the TEC hot side is oriented away from the patient's head and makes contact with a heat dissipation system.

80. The sysiem of clause 79 wherein the heat dissipation system may be a thermally conductive fluid that flows across the hot side of the TEC or a layer covering the TEC where the fluid absorbs the heat from the TEC and moves it to a heatsink or fan that removes the heat for dissipation to atmosphere.

81. The system of clause 79 wherein the heat dissipation system may be a heatsink or fansink made from a metallic or ceramic material and designed to accept heat from the hot side of the TEC and dissipate said heat to atmosphere via natural convection or forced convection.

82. The system of clause 86 wherein said temperature sensing unit may be configured to use invasive or non-invasive temperature sensors to monitor the temperature of the brain, skin, eye, ear, and core of the patient.

83. The system of clause 82 wherein said temperature sensing system may be configured to have temperature sensors that interface with the patient's head, neck, and body that provide temperature information to the power control system in an open or closed feedback loop.

84. The system of clause 83 wherein said temperature sensors may be thermistors, thermocouples, RTDs, thermal imaging systems, or microwave emission sensors that may be used individually or in any combination thereof.

85. The system of clause 84 wherein said temperature sensors may be attached to the headliner, neck brace, to the patient's skin, face, ear, or body or be configured not to make direct physical contact with the body such as with an optical or thermal imaging system.

86. The system of clause 17 wherein said temperature sensors may be configured to sense temperature remotely and not be physically attached to the body but be configured to measure temperature indirectly such as with a thermal imaging camera.

87. The system of clause 68 wherein said power control system is configured to received electrical power from a power supply that may receive alternating current, direct current, power from primary batteries, or power from secondary batteries.

88. The system of clause 87 wherein said power control system is configured to regulate the power delivered to the source of heat transfer located on the headliner, neck brace, or both simultaneously.

89. The system of clause 88 wherein said power control system is configured to received feedback from the temperature sensing system and create a closed loop feedback system to regulate the power supplied to the source of heat transfer for the headliner, neck brace, or both simultaneously.

90. The system of clause 89 wherein said power control system may be configured to allow for set temperature ranges, time to reach set temperature range, maximum and minimum safe operating temperatures, alarms if a set temperature leaves its programmed range, alarms for battery life, and pre-programed safety protocols.

91. The system of clause 68 wherein a neck brace is used to support and stabilize the head and neck of the patient, said neck brace is designed to surround the neck of the patient and provide support to the neck, cervical spine, and head of the patient.

92. The system of clause 9 wherein said neck brace is designed to interface with the headliner and be configured to allow a source of heat transfer and a heat transfer material to be configured into the neck brace and be configured to be in a heat transfer relationship with the head and neck of the patient.

93. The system of clause 92 wherein said neck brace being made of multiple components so that the neck brace may be of a universal fit and configured to fit around the patient's neck and adjusted to support and stabilize the head and neck of the patient.

94. The system of clause 92 wherein said neck brace is configured to house multiple sources of heat transfer such as a thermoelectric cooling element that is configured for the cold side to face the patient and the hot side to face away from the body.

95. The system of clause 94 wherein said heat transfer source and heat transfer material in the neck brace has a different temperature than that of the patient's head and neck, such that heat is transferred between the heat transfer material and heat transfer source and the head and neck of the patient.

96. The system of clause 94 wherein said thermoelectric cooling elements are configured to interface with a heat dissipation system that removes heat from the hot side of the thermoelectric cooling element and dissipates that heat to atmosphere.

97. The system of clause 96 wherein said heat dissipation system may be configured to use a heatsink, fan and heatsink combination, heat pipes, phase change material, or a gas or fluid cooling system.

98. The system of clause 93 wherein said neck brace is configured to interface with the headliner and may use straps that are elastic in nature, such that the headliner is caused to be held to the head of the patient by the tension created by the elastic nature to allow for a secure and customizable fit to the patient's head.

99. The system of clause 68 wherein said headliner and neck brace are configured to be contained in an envelope that is sanitary and disposable, wherein said headliner and neck brace are also disposable after use.

We claim:

1. A selective brain cooling system comprising:
a headliner configured for disposition on a head; and
two or more first thermoelectric cooling elements (TECs) mounted to the headliner.

2. The system of claim 1, the headliner comprising two or more segments, each segment having at least one of the first TECs.

3. The system of claim 1, the headliner comprising two or more bands, each band comprising two or more segments, each segment having at least one of the first TECs.

4. The system of claim 1, the headliner comprising a plurality of patches, each patch having at least one of the first TECs.

5. The system of claim 1 further comprising:
a neck brace configured for disposition on a neck; and
two or more second TECs mounted to the neck brace.

6. The system of claim 1 further comprising:
a power supply electrically coupled to the first TECs so as to provide electrical power thereto; and
a power controller electrically coupled between the power supply and the first TECs to control the electrical power provided by the power supply to the first TECs.

7. The system of claim 6 further comprising a temperature sensor configured to sense the temperature of the head or a portion thereof.

8. The system of claim 7, wherein the temperature sensor provides temperature data to the power controller, the power controller being configured to control the electrical power in response to the temperature data.

9. The system of claim 8, the temperature sensor being separate from the headliner.

10. The system of claim 8, the headliner further comprising the temperature sensor.

11. The system of claim 6, the power supply comprising a battery, or wall outlet electrical power, or both.

12. The system of claim 1, the headliner being a cap configured to cover at least part of the head.

13. The system of claim 12, the headliner comprising removable segments.

14. The system of claim 12, the headliner comprising removable bands.

15. The system of claim 1, the headliner being configured to cover all or part of the scalp.

16. The system of claim 1, the headliner being configured to cover all or part of the face.

17. The system of claim 1, the headliner being configured to cover all or part of the scalp and the face.

18. The system of claim 1, the headliner comprising two or more portions, each portion being configured to cover a part of the head.

19. The system of claim 18, a first portion configured to cover the back of the head, a second portion configured to cover the top of the head, a third portion configured to cover a first side of the head, and a fourth portion configured to cover a second side of the head.

20. The system of claim 19, the third portion and the fourth portion each being configured to fit about an ear.

21. The system of claim 1, the headliner being configured to allow physical removal of at least one of the first TECs.

22. The system of claim 1, the headliner being configured to allow physical re-orientation of at least one of the first TECs.

23. The system of claim 1, each of the first TECs being mounted to the headliner so as to place the cold side of the TEC toward the head and the hot side of the TEC away from the head.

24. The system of claim 23, further comprising two or more heat sinks, each heat sink being thermally coupled to at least one of the first TECs.

25. The system of claim 23, the headliner comprising a thermally-conductive layer, the cold side of each of the first TECs being disposed against the thermally-conductive layer.

26. The system of claim 1, the headliner configured for adhesive disposition on the head.

27. The system of claim 1, the headliner configured for compressive disposition on a head.

28. The system of claim 1, the headliner comprising a frame configured to allow placement of one or more of the first TECs on the head so as to minimize pressure to the head.

29. A selective brain cooling system comprising:
a neck brace configured for disposition on a neck; and
two or more thermoelectric cooling elements (TECs) mounted to the neck brace.

30. The system of claim 29, the neck brace comprising a flexible wrap configured to cover all or part of the neck.

31. The system of claim 29, the neck brace comprising a structure configured to support the neck and head.

32. The system of claim 29, the neck brace comprising two or more segments, each segment having at least one of the TECs.

33. The system of claim 29, the neck brace comprising two or more bands, each band comprising two or more segments, each segment having at least one of the TECs.

34. The system of claim 29, the neck brace comprising a plurality of patches, each patch having at least one of the TECs.

35. The system of claim 29 further comprising:
a power supply electrically coupled to the TECs so as to provide electrical power thereto; and
a power controller electrically coupled between the power supply and the TECs to control the electrical power provided by the power supply to the TECs.

36. The system of claim 35 further comprising a temperature sensor configured to sense the temperature of the neck or a portion thereof.

37. The system of claim 36, wherein the temperature sensor provides temperature data to the power controller, the power controller being configured to control the electrical power in response to the temperature data.

38. The system of claim 37, the temperature sensor being separate from the neck brace.

39. The system of claim 37, the headliner further comprising the temperature sensor.

40. The system of claim 35, the power supply comprising a battery, or wall outlet electrical power, or both.

41. The system of claim 29, the neck brace being configured to cover at least part of the neck.

42. The system of claim 41, the neck brace comprising removable segments.

43. The system of claim 41, the neck brace comprising removable bands.

44. The system of claim 29, the neck brace comprising two or more portions, each portion being configured to cover a part of the neck.

45. The system of claim 44, a first portion configured to cover the back of the neck, a second portion configured to cover the front of the neck.

46. The system of claim 44, a first portion configured to cover a first carotid artery of the neck, a second portion configured to cover a second carotid artery of the neck.

47. The system of claim 29, the neck brace being configured to allow physical removal of at least one of the TECs.

48. The system of claim 29, the headliner being configured to allow physical re-orientation of at least one of the TECs.

49. The system of claim 29, each of the first TECs being mounted to the neck brace so as to place the cold side of the TEC toward the neck and the hot side of the TEC away from the neck.

50. The system of claim 49, further comprising two or more heat sinks, each heat sink being thermally coupled to at least one of the TECs.

51. The system of claim 49, the neck brace comprising a thermally-conductive layer, the cold side of each of the TECs being disposed against the thermally-conductive layer.

52. The system of claim 29, the neck brace configured for adhesive disposition on the neck.

53. The system of claim 29, the neck brace configured for compressive disposition on a neck.

54. The system of claim 29, the neck brace comprising a frame configured to support the head and neck, and to allow placement of one or more of the TECs on the neck so as to minimize pressure to the neck.

55. A method of selectively cooling a brain, the method comprising:
disposing a headliner on or about a head, the headliner comprising two or more thermoelectric cooling elements (TECs); and
electrically activating at least one of the TECs.

56. The method of claim 55, further comprising controlling the electrical activation of the TECs with a power controller.

57. The method of claim 55, further comprising configuring the headliner for disposition over one or more parts of the head.

58. The method of claim 57, wherein the headliner comprises one or more segments, each segment including at least one of the TECs, and the configuring comprises removing a segment from the headliner.

59. The method of claim 57, wherein the headliner comprises one or more bands, each band including at least two of the TECs, and the configuring comprises removing a band from the headliner.

60. A method of selectively cooling a brain, the method comprising:
disposing a neck brace on or about a neck, the headliner comprising two or more thermoelectric cooling elements (TECs); and
electrically activating at least one of the TECs.

61. The method of claim 60, further comprising controlling the electrical activation of the TECs with a power controller.

62. The method of claim 60, further comprising configuring the neck brace for disposition over one or more parts of the neck.

63. The method of claim 62, further comprising disposing the neck brace over at least one of the carotid arteries.

64. The method of claim 62, wherein the neck brace comprises one or more segments, each segment including at least one of the TECs, and the configuring comprises removing a segment from the neck brace.

65. The method of claim 62, wherein the neck brace comprises one or more bands, each band including at least two of the TECs, and the configuring comprises removing a band from the neck brace.

66. A headliner comprising one or more thermoelectric cooling elements.

67. A neck brace comprising one or more thermoelectric cooling elements.

68. A portable selective brain cooling system for controlling the temperature of a patient's brain and brain stem by controlling head and neck temperature, the system comprising:
a source of heat transfer; and
a heat transfer material; and
a headliner; and
a temperature sensing system; and
a heat dissipation system; and
a neck brace; and
a power control system,
said headliner having flexible and adjustable segments being of a size to fit around the head of the patient leaving the face and ears exposed,
said headliner being flexible and configurable to be folded to a smaller size with a flat orientation, and a secondary orientation where the headliner is adjustable to be placed downward or around the head in a manner that allows for the majority of the head to be covered by the headliner except for the face and ears,
said headliner may have a fluid, phase change material, or high thermal conductivity material inside the headliner segments, where the source of heat transfer makes contact with the high thermal conductivity material and said material is in contact with the inner surface of the headliner that will make contact with the surface of the patient's head,
wherein said heat transfer material has a different temperature than the head of the patient and is arranged to be in contact with the source of heat transfer and the inner surface of the headliner such that heat is transferred from the patient's heat to the heat transfer material and to the heat transfer source.

69. The system of claim 68 wherein said heat transfer material interfacing with said heat transfer source is selected to be cooler than the temperature of the head of the patient when the heat transfer source is activated by the power control system, the power control and temperature control systems being configured to be attached to or located within the headliner.

70. The system of claim 69 wherein said heat transfer source may comprise of a single or multiple thermoelectric cooling elements (TEC), said TEC being part of a cooling assembly designed to interface with the headliner and the heat transfer material inside the headliner, said TEC being oriented so that the cold side of the TEC is oriented towards the heat transfer material and the hot side of the TEC is oriented away from the patient's head, said TEC being interfaced with a heat dissipation system that dissipates heat from the TEC to atmosphere.

71. The system of claim 68 where said headliner includes either adhesive or elastic straps attached to the outward facing surface of the headliner, overlying multiple segments of the headliner, adapted such that the adhesive or elastic straps can be used to tighten or loosen the fit of the headliner on the patient's head to adjust contact surface area between the headliner and the patient's head and provide a secure interface.

72. The system of claim 71 wherein said headliner includes at least three layers, an inner layer that makes contact with the patient's head and the thermally conductive middle layer, the thermally conductive middle layer that makes contact with the inner layer and outer layers of the headliner, the outer layer that interfaces with the middle layer and the source of heat transfer, the source of heat transfer that interfaces with the outer layer and the heat dissipation system, where the heat dissipation system interfaces with the atmosphere.

73. The system of claim 72 wherein said headliner inner and outer layers may be made of a polymeric material that is selected from the group of silicone, urethane, silicone-polyurethane block copolymer, and a thermoplastic elastomer.

74. The system of claim 72 wherein said headliner middle layer may be made of a thermally conductive material such as phase change material, thermally conductive fluids, or a thermally enhanced polymeric material.

75. The system of claim 70 wherein said headliner includes a single layer where the inner surface interfaces with the patient's head and the outer layer interfaces with the heat transfer source.

76. The system of claim 75 wherein said headliner may be made of a polymeric material selected from the group of silicone, urethane, silicone-polyurethane block copolymer, and a thermoplastic elastomer.

77. The system of claim 76 wherein the polymeric material may be made to enhance the thermal conductivity thereof, via a highly thermally conductive additive dispersed within the polymeric material.

78. The system of claim 75 wherein the headliner may be made of a fabric material that is modified to enhance the thermal conductivity thereof through the use of one or more mechanisms selected from the group of conductive coatings, conductive filler doping, phase change materials, knit-in or wound wires, thermally conductive powders, and combinations thereof.

79. The system of claim 70 wherein the headliner may be made of an inner layer that interfaces with the head and the source of heat transfer such as a TEC, where the TEC cold side is oriented towards the patient's head and makes contact with the inner layer of the headliner, where the TEC hot side is oriented away from the patient's head and makes contact with a heat dissipation system.

80. The system of claim 79 wherein the heat dissipation system may be a thermally conductive fluid that flows across the hot side of the TEC or a layer covering the TEC where the fluid absorbs the heat from the TEC and moves it to a heatsink or fan that removes the heat for dissipation to atmosphere.

81. The system of claim 79 wherein the heat dissipation system may be a heatsink or fansink made from a metallic or ceramic material and designed to accept heat from the hot side of the TEC and dissipate said heat to atmosphere via natural convection or forced convection.

82. The system of claim 86 wherein said temperature sensing unit may be configured to use invasive or non-invasive temperature sensors to monitor the temperature of the brain, skin, eye, ear, and core of the patient.

83. The system of claim 82 wherein said temperature sensing system may be configured to have temperature sensors that interface with the patient's head, neck, and body that provide temperature information to the power control system in an open or closed feedback loop.

84. The system of claim 83 wherein said temperature sensors may be thermistors, thermocouples, RTDs, thermal imaging systems, or microwave emission sensors that may be used individually or in any combination thereof.

85. The system of claim 84 wherein said temperature sensors may be attached to the headliner, neck brace, to the patient's skin, face, ear, or body or be configured not to make direct physical contact with the body such as with an optical or thermal imaging system.

86. The system of claim 17 wherein said temperature sensors may be configured to sense temperature remotely and not be physically attached to the body but be configured to measure temperature indirectly such as with a thermal imaging camera.

87. The system of claim 68 wherein said power control system is configured to received electrical power from a power supply that may receive alternating current, direct current, power from primary batteries, or power from secondary batteries.

88. The system of claim 87 wherein said power control system is configured to regulate the power delivered to the source of heat transfer located on the headliner, neck brace, or both simultaneously.

89. The system of claim 88 wherein said power control system is configured to received feedback from the temperature sensing system and create a closed loop feedback system to regulate the power supplied to the source of heat transfer for the headliner, neck brace, or both simultaneously.

90. The system of claim 89 wherein said power control system may be configured to allow for set temperature ranges, time to reach set temperature range, maximum and minimum safe operating temperatures, alarms if a set temperature leaves its programmed range, alarms for battery life, and pre-programed safety protocols.

91. The system of claim 68 wherein a neck brace is used to support and stabilize the head and neck of the patient, said neck brace is designed to surround the neck of the patient and provide support to the neck, cervical spine, and head of the patient.

92. The system of claim 9 wherein said neck brace is designed to interface with the headliner and be configured to allow a source of heat transfer and a heat transfer material to be configured into the neck brace and be configured to be in a heat transfer relationship with the head and neck of the patient.

93. The system of claim 92 wherein said neck brace being made of multiple components so that the neck brace may be of a universal fit and configured to fit around the patient's neck and adjusted to support and stabilize the head and neck of the patient.

94. The system of claim 92 wherein said neck brace is configured to house multiple sources of heat transfer such as a thermoelectric cooling element that is configured for the cold side to face the patient and the hot side to face away from the body.

95. The system of claim 94 wherein said heat transfer source and heat transfer material in the neck brace has a different temperature than that of the patient's head and neck, such that heat is transferred between the heat transfer material and heat transfer source and the head and neck of the patient.

96. The system of claim 94 wherein said thermoelectric cooling elements are configured to interface with a heat dissipation system that removes heat from the hot side of the thermoelectric cooling element and dissipates that heat to atmosphere.

97. The system of claim 96 wherein said heat dissipation system may be configured to use a heatsink, fan and heatsink combination, heat pipes, phase change material, or a gas or fluid cooling system.

98. The system of claim 93 wherein said neck brace is configured to interface with the headliner and may use straps that are elastic in nature, such that the headliner is caused to be held to the head of the patient by the tension created by the elastic nature to allow for a secure and customizable fit to the patient's head.

99. The system of claim 68 wherein said headliner and neck brace are configured to be contained in an envelope that is sanitary and disposable, wherein said headliner and neck brace are also disposable after use.
